(19) United States
(12) Patent Application Publication
(10) Pub. No.: US 2026/0283933 A1
(43) Pub. Date: Sep. 24, 2026

(54) OIL-IN-WATER COSMETIC

(71) Applicant: SHISEIDO COMPANY, LTD, Tokyo (JP)

(72) Inventors: Yudi SUN, Tokyo (JP); Toru OKAMOTO, Tokyo (JP)

(73) Assignee: SHISEIDO COMPANY, LTD, Tokyo (JP)

(21) Appl. No.: 19/479,976

(22) PCT Filed: May 16, 2024
(86) PCT No.: PCT/JP2024/018085

§ 371 Date: Oct. 29, 2025

(30) Foreign Application Priority Data
May 29, 2023 (JP) 2023-087799

(51) Int. Cl. A61K 8/86; A61K 8/06; A61K 8/31; A61K 8/34; A61K 8/37; A61K 8/44; A61K 8/46; A61K 8/49; A61K 8/891; A61Q 17/04; A61Q 19/00

(52) U.S. Cl. CPC A61K 8/86; A61K 8/062; A61K 8/31; A61K 8/34; A61K 8/345; A61K 8/37; A61K 8/375; A61K 8/442; A61K 8/466; A61K 8/4966; A61K 8/891; A61Q 17/04; A61Q 19/007; A61K 2800/413

(57) ABSTRACT

The present invention provides an oil-in-water cosmetic having excellent moisture-retaining property and high transparency. A cosmetic according to the present invention is an oil-in-water cosmetic containing (A1) a first surfactant of a specific configuration containing an alkyleneoxy group having 3 or 4 carbon atoms and an ethyleneoxy group, (A2) a second surfactant selected from ionic surfactants, (B1) a polar oil, and (C) water, in which
the content rate of component (B) to the total mass of oil component contained in said oil-in-water cosmetic is 50 mass % or more, and
the ratio B/A1 of the total mass of oil component (B) to the total mass of component (A1) contained in said oil-in-water cosmetic, is 1 to 5.

### TECHNICAL FIELD

The present invention relates to a transparent oil-in-water emulsion cosmetic that can exhibit excellent moisture-retaining property.

### BACKGROUND ART

Creamy cosmetics and milky lotions having an excellent moisture-retaining property are often used for preventing dry skin in winter. However, applying a cosmetic having a relatively high viscosity to the skin often requires manipulation such as spreading the cosmetic by hand. To make it easier to apply than cream-type cosmetics, cosmetics that can be sprayed in the form of a mist have also been developed. It is generally desired for such a cosmetics that can be sprayed in the form of a mist to have high transparency.

In contrast, it is desired to add another function to such a cosmetic in combination with a moisture-retaining property. To be more specific, it is also desired for cosmetics to provide, e.g., an effect of protecting the skin from UV simultaneously with an effect of making the skin glossy. Such a cosmetic that can provide effects in combination, since it can be easily used before going out, and even outside the home, is easily accepted by consumers.

In contrast, it is desired for cosmetics that are directly applied to the skin to provide a comfortable feeling of use such as freshness, moist feeling, and fine texture, at the time of application. Various cosmetics have been studied in order to realize such a feeling of use, but it is known that cosmetics having a high content rate of water are excellent since freshness can be provided. In order to blend a functional oily ingredient in such a cosmetic, an oil-in-water cosmetic is properly used.

However, if an effective oily ingredient is blended at a high ratio, the transparency of the cosmetic may sometimes decrease. From this perspective, components of cosmetics and emulsification techniques have been investigated, but cosmetics that can provide not only a high moisture-retaining property but also high transparency are desired.

### CITATION LIST

### Patent Literature

Patent Literature 1: JP 2011-157282A

### SUMMARY OF THE INVENTION

An object of the present invention is to provide an oil-in-water cosmetic that can provide not only a high moisture-retaining property but also high transparency and can further produce effects other than a moisture-retaining property.

According to the present invention, the following cosmetics are provided.

[1]

An oil-in-water cosmetic including:
(A1) a first surfactant represented by the following formula (1):

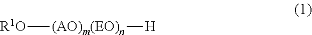

wherein
R¹ is a hydrocarbon group having 16 to 18 carbon atoms,
AO is an alkyleneoxy group having 3 or 4 carbon atoms,
EO is an ethyleneoxy group,
AO and EO are each added in block form,
m and n represent average numbers of moles of AO and EO added, respectively,

$$\begin{matrix}m > 4 , \\ n > 10 , and \\ n > m ,\end{matrix}$$

(A2) a second surfactant selected from ionic surfactants,
(B1) a polar oil, and
(C) water,
wherein
a content rate of component (B1) to a total mass of oil component (B) contained in the oil-in-water cosmetic is 50 mass % or more, and
a ratio B/A1 of the total mass of oil component (B) to a total mass of component (A1) contained in the oil-in-water cosmetic is 1 to 5.

[2]

The oil-in-water cosmetic according to [1], wherein component (A2) is a compound containing two carboxyl groups.

[3]

The oil-in-water cosmetic according to [1] or [2], wherein component (A2) comprises a structure represented by the following Formula (2):

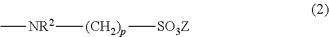

wherein
R² is H or a hydrocarbon group having 1 or 2 carbon atoms,
Z is H, Na, K, or NR^{a}₄ (wherein R^{a}s are each independently H, CH₃, C₂H₅, CH₂OH or CH₂CH₂OH), and
p is 1 to 3.

[4]

The oil-in-water cosmetic according to any one of [1] to [3], wherein at least a part of component (B1) is a UV absorber.

[5]

The oil-in-water cosmetic according to any one of [1] to [4], wherein a content rate of component (A1) to a total mass of the oil-in-water cosmetic is 0.1 to 15 mass %.

[6]

The oil-in-water cosmetic according to any one of [1] to [5], wherein a content rate of component (A2) to the total mass of the oil-in-water cosmetic is 0.01 to 1 mass %.

[7]

The oil-in-water cosmetic according to any one of [1] to [6], wherein a ratio A2/A1 of a total mass of component (A2) to a total mass of component (A1) contained in the oil-in-water cosmetic is 0.01 to 0.05.

[8]

The oil-in-water cosmetic according to any one of [1] to [7], further comprising a polyhydric alcohol (D).

[9]

The oil-in-water cosmetic according to [8], wherein a content rate of component (D) to the total mass of the cosmetic is 3 to 30 mass %.

The oil-in-water cosmetic according to any one of [1] to [9], wherein oil droplet particles contained in the oil-in-water cosmetic have an average particle diameter of 70 nm or less.

According to the present invention, there is provided a cosmetic having not only a high moisture-retaining property but also high transparency. Since the content of an oily ingredient can be increased in the cosmetic, oily ingredients having other functions can be blended while maintaining high transparency. For example, a UV absorbing function can be added.

### DETAILED DESCRIPTION OF THE INVENTION

An oil-in-water cosmetic (hereinafter, sometimes referred to simply as a “cosmetic”) according to the present invention includes:
(A1) a first surfactant,
(A2) a second surfactant,
(B1) a polar oil, and
(C) water. Individual components contained in the cosmetic according to the present invention are as follows.

### (A) Surfactant

The cosmetic according to the present invention contains a surfactant (hereinafter, sometimes referred to as component (A)). Surfactants can be classified into several types, but the cosmetic according to the present invention contains a first surfactant and a second surfactant in combination.

### (A1) First Surfactant

The first surfactant (hereinafter, sometimes referred to as component (A1)) to be used in the cosmetic according to the present invention is represented by the following Formula (1):

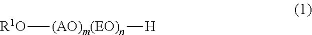

wherein:
R¹ is a hydrocarbon group having 16 to 18 carbon atoms,
AO is an alkyleneoxy group having 3 or 4 carbon atoms,
EO is an ethyleneoxy group,
AO and EO are each added in block form, and
m and n represent average numbers of moles of AO and EO added, respectively,

$$\begin{matrix}m > 4 , \\ n > 10 , and \\ n > m .\end{matrix}$$

Component (A1) is a nonionic surfactant also called as a polyalkylene alkyl ether, as is apparent from Formula (1). The surfactant contains both a polyoxyethylene block and another block, i.e., a polyoxyalkylene such as polyoxypropylene.

The weight average molecular weight of component (A1) represented by Formula (1) is preferably 800 or more and more preferably 1500 or more. If the weight average molecular weight is less than 800, the effect is low. The upper limit of the weight average molecular weight is not particularly limited, but as the weight average molecular weight increases, sticky feeling may be produced and thus care must be taken.

As component (A1) represented by Formula (1), for example, a commercially available product such as NIKKOL PBC44 (manufactured by Nikko Chemicals Co., Ltd., R¹═CH₃ (CH₂)₁₅, m=8, n=20) can be used.

In the present invention, component (A1) as well as a second surfactant (described later) principally provide an effect of improving dispersion stability of an oil component (described later). Accordingly, the content rate of component (A1) may vary depending on the content of the oil component, but the content rate of component (A1) to the total mass of the cosmetic is preferably 0.1 to 15 mass %, more preferably 0.5 to 10 mass %, and particularly preferably 1 to 7 mass %.

### (A2) Second Surfactant

The cosmetic according to the present invention contains, as a second surfactant (hereinafter, sometimes referred to as component (A2)), a surfactant selected from ionic surfactants. Component (A2) may be any one of an anionic surfactant, a cationic surfactant, and an amphoteric surfactant. Of them, an anionic surfactant is preferably used because the effects of the present invention tend to be more strongly exhibited.

As the anionic surfactant, various types of surfactants are known. Examples thereof include
(i) a carboxylic acid type anionic surfactant having a carboxyl group (—COOH) or a carboxylate group (—COO—),
(ii) a sulfonic acid type anionic surfactant containing a sulfo group (—SO₃H) or a sulfonate (—SO₃⁻) group, and
(iii) a sulfate type anionic surfactant containing a sulfate group (—O—SO₃⁻). Of these anionic surfactants, surfactants exerting an effect as a surfactant in a neutral region of pH8 or less are preferable in the present invention.

For example, as the (i) carboxylic acid type anionic surfactant, various surfactants are known, but in the present invention, component (A2) is preferably a compound containing two carboxyl groups rather than a basic anionic surfactant such as a higher fatty acid soap. Specifically, disodium N-stearoyl-L-glutamate, or disodium N-octanoyl glutamate can be used.

In the present invention, when (ii) a sulfonic acid type anionic surfactant is used as component (A2), it is preferable to use a compound containing a structure represented by the following Formula (2):

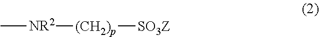

wherein:
R² is H or a hydrocarbon group having 1 or 2 carbon atoms,
Z is H, Na, K, or NR^{a}₄ (wherein R^{a}s are each independently H, CH₃, C₂H₅, CH₂OH or CH₂CH₂OH), and
p is 1 to 3. Herein, in order to increase the water solubility of component (A2), Ra is preferably H, C₂H₅, CH₂OH, or CH₂CH₂OH. To be more specific,
it is possible to use, for example,
sodium stearoyl methyl taurate,
Na methyl cocoyl taurate,
Na taurine cocoyl methyltaurate
cocoyl-N-methyl taurate lauryl methyl taurate
myristyl methyl taurate,
sodium cocoyl taurate, or
sodium cocoyl isethionate.

In the present invention, component (A2) as well as component (A1) principally provide an effect of improving dispersion stability of an oil component (described later). Therefore, the content rate of component (A2) may vary depending on the content rate of component (A1) and the content rate of the oil component, but the content rate of component (A2) to the total mass of the cosmetic is preferably 0.01 to 1 mass % and more preferably 0.05 to 0.5 mass %.

Also, the cosmetic according to the present invention contains component (A1) in combination with component (A2), but the dispersion stability of the cosmetic may vary depending on the blending ratio of these. Because of this, the ratio A2/A1 of the total mass of component (A2) to the total mass of component (A1) contained in the cosmetic is preferably 0.01 to 0.05.

### (B) Oil Component

The cosmetic according to the present invention contains an oil component (hereinafter, sometimes referred to as component (B)). Since the cosmetic according to the present invention is an oil-in-water cosmetic containing an oil component, it is possible to blend an oily ingredient or an oil-soluble ingredient that can provide a function to the cosmetic.

The oil components can be classified as polar oils (hereinafter, sometimes referred to as component (B1)) and non-polar oils (hereinafter, sometimes referred to as component (B2)). The cosmetic according to the present invention must contain component (B1). The content rate of component (B1) to the total mass of component (B) contained in the oil-in-water cosmetic is 50 mass % or more. The content rate of component (B1) to the total mass of component (B) contained in the oil-in-water cosmetic is preferably 70 mass % or more. Since the cosmetic according to the present invention may not contain component (B2), component (B) may only consist of component (B1). Hereinafter, component (B1) and component (B2) will be described.

### (B1) Polar Oil

The cosmetic according to the present invention contains a polar oil. In the present invention, the polar oil refers to an oil having a polar group, that is, a hydrophilic group. The polar oil can be arbitrarily selected from those commonly used in, e.g., the cosmetic field, the pharmaceutical field and the food field depending on the desirable function for cosmetics. Of these, a polar oil having an IOB value of 0.05 to 0.80 is preferable. The IOB value, which is an abbreviation of Inorganic/Organic Balance (inorganic/organic ratio), is a value representing the ratio of the inorganic value to the organic value, and is used as an index indicating the degree of polarity of the organic compound. The IOB value is specifically represented by the equation:

IOB value=inorganic value/organic value. Herein, the “inorganic value” and the “organic value” are defined as follows: the “inorganic value” and the “organic value” are defined in accordance with the types of atoms or functional groups, more specifically, the “organic value” per carbon atom in a molecule is specified as 20, whereas the “inorganic value” per hydroxy group in a molecule is specified as 100. The IOB value of the organic compound can be calculated by integrating the “inorganic values” and the “organic values” of all atoms and functional groups in an organic compound.

In the present invention, a polar oil that can provide a function to a cosmetic product can be used. Specifically, it is possible to use a polar oil that can make the skin glossy, colored, and textured and provide, e.g., a UV absorption effect and a protective film forming effect, on the skin, when a cosmetic product is personally used.

Examples of the polar oil include ester oils, higher fatty acids, higher alcohols, and waxes. Of these, an ester oil is preferably used. Of the polar oils, one having a high UV absorption effect is generally called a UV absorber, and can be used to provide a function of protecting the skin from UV rays to the cosmetic product according to the present invention. The cosmetic according to the present invention preferably contains such a UV absorber as component (B1).

Examples of the ester oil include glyceryl (behenate/eicosanedioate), tripropylene glycol dineopentanoate, isononyl isononanoate, isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, cetyl ethylhexanoate, ethylene glycol di-2-ethylhexanoate, dipentaerythrityl fatty acid ester, n-alkylglycol mono-isostearate, neopentyl glycol dicaprate, diisostearyl malate, glycerin di-2-heptylundecanoate, trimethylolpropane tri-2-ethylhexanoate, trimethylolpropane triisostearate, pentaerythrityl tetra-2-ethylhexanoate, triethylhexanoin (glycerin tri-2-ethylhexanoate), glycerin trioctanoate, glycerin triisopalmitate, trimethylolpropane triisostearate, cetyl 2-ethylhexanoate, 2-ethylhexylpalmitate, glycerin trimyristate, glyceride tri-2-heptylundecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, and triethyl citrate.

The UV absorber is not particularly limited, but a wide variety of UV absorbers commonly used in cosmetics can be mentioned. For example,
(a1) benzoic acid derivatives such as ethyl para-aminobenzoate (PABA), ethyl-dihydroxypropyl PABA, ethylhexyl-dimethyl PABA, glyceryl PABA, PEG-25 PABA, and diethylamino hydroxybenzoyl hexyl benzoate;
(a2) salicylic acid derivatives such as homosalate, ethylhexyl salicylate (octyl salicylate), dipropylene glycol salicylate, and TEA salicylate;
(a3) cinnamic acid derivatives such as octyl methoxycinnamate, ethylhexyl methoxycinnamate, isopropyl methoxycinnamate, isoamyl methoxycinnamate, cinnoxate, DEA methoxycinnamate, diisopropyl methylcinnamate, glyceryl-ethylhexanoate-dimethoxycinnamate, and di-(2-ethylhexyl)-4′-methoxybenzalmalonate;
(a4) dibenzoylmethane derivatives such as 4-tert-butyl-4′-methoxydibenzoylmethane;
(a5) β,β-diphenylacrylate derivatives such as octocrylene;
(a6) benzophenone derivatives such as benzophenone-1, benzophenone-2, benzophenone-3 or oxybenzone, benzophenone-4, benzophenone-5, benzophenone-6, benzophenone-8, benzophenone-9, and benzophenone-12;
(a7) benzylidene camphor derivatives such as 3-benzylidene camphor, 4-methylbenzylidene camphor, benzylidene camphor sulfonic acid, camphor benzalkonium methosulfate, terephthalylidene dicamphor sulfonic acid, and polyacrylamide methylbenzylidene camphor;
(a8) phenylbenzimidazole derivatives such as phenylbenzimidazole sulfonic acid, and disodium phenylbenzimidazole tetrasulfonate;
(a9) triazine derivatives such as bisethylhexyloxyphenol methoxyphenyltriazine, ethylhexyltriazone (e.g., “Uvinal T150”; BASF), diethylhexylbutamidotriazone, 2,4,6-tris(diisobutyl-4′-aminobenzalmalonate)-s-triazine, and 2,4,6-tris[4-(2-ethylhexyloxycarbonyl) anilino]-1,3,5-triazine;
(a10) phenylbenzotriazole derivatives such as drometrizole trisiloxane, and methylenebis(benzotriazolyltetramethylbutylphenol);
(a11) anthranil derivatives such as menthyl anthranilate;
(a12) imidazoline derivatives such as ethylhexyl dimethoxybenzylidene dioxoimidazoline propionate;
(a13) benzalmalonate derivatives such as a polyorganosiloxane having a benzalmalonate functional group; and
(a14) 4,4-diarylbutadiene derivatives such as 1,1-dicarboxy (2,2′-dimethylpropyl)-4,4-diphenylbutadiene.

In the present invention, the polar oil can be arbitrarily selected from these and put in use. Two or more types of polar oils may be used in combination. Some of the polar oils shown as examples are present in solid form such as powder at normal temperature and may be dissolved in other polar oils and put in use. The cosmetic according to the present invention preferably contains a UV absorber as the polar oil. The cosmetic according to the present invention containing a UV absorber as the polar oil, is preferable as a sun-care cosmetic.

In the cosmetic according to the present invention, the content rate of component (B1) is relatively high in order to sufficiently produce an aesthetic effect and a UV protection effect. Specifically, the content rate of component (B1) is preferably 0.5 to 20 mass % and more preferably 3 to 15 mass % based on the total mass of the cosmetic. When the content rate of a polar oil is low, the function to be realized by the polar oil may not be sufficiently produced. Thus, care must be taken.

In addition, in order to maintain high dispersion stability of the cosmetic, the ratio B/A1 of the total mass of oil component (B) to the total mass of component (A1) contained in the cosmetic is 1 to 5. Since dispersibility can be further improved, B/A1 is preferably 2 to 4 and more preferably 2.5 to 3.5.

### (B2) Non-Polar Oil

The cosmetic according to the present invention may also contain a non-polar oil. The non-polar oil is an oil component except component (B1) and having an IOB value of less than 0.05. Examples of the non-polar oil include:
(i) hydrocarbon oils such as liquid paraffin, squalane, squalene, paraffin, and isohexadecane, and
(ii) silicone oils, for example, linear silicone oils such as dimethylpolysiloxane, methylphenylpolysiloxane, caprylyl methicone, diphenylsiloxy phenyl trimethicone, and methylhydrogen polysiloxane; and silicone cyclic oils such as octamethylcyclotetrasiloxane, decamethylcyclopentasiloxane, and dodecamethylcyclohexasiloxane.

### (C) Water

The composition according to the present invention further contains water in addition to the above components. As the water, water used for, e.g., cosmetic products and quasi-drugs, can be used. Examples of the water that can be used include purified water, ion-exchanged water, and tap water.

In the cosmetic according to the present invention, the content rate of water based on the total mass of the cosmetic is generally 30 to 95 mass % and preferably 40 to 80 mass % based on the total mass of the cosmetic.

### (D) Polyhydric Alcohol

The composition according to the present invention may contain, in addition to the essential components described above, a polyhydric alcohol (hereinafter, sometimes referred to as component (D)). Component (D) produces a function of further improving the moisturizing effect by the oil-in-water cosmetic according to the present invention. Examples of the polyhydric alcohol include glycerin, 1,3-butylene glycol, ethylene glycol, propylene glycol, dipropylene glycol, polyethylene glycol, polypropylene glycol, polyethylene glycol⋅polypropylene glycol, trimethylolethane, trimethylolpropane, erythritol, pentaerythritol, sorbitan, glucose, sorbitol, maltitol, sucrose, raffinose, hexylene glycol, 1,2-pentanediol, and trehalose.

When the cosmetic according to the present invention contains component (D), the content rate of component (D) to the total mass of the cosmetic is preferably 3 to 30 mass %.

### (E) Other Components

The cosmetic according to the present invention may contain various additives as necessary in addition to the essential components described above.

One of these additives is an alkylene oxide derivative. The alkylene oxide derivative can improve a moisturized feel of the skin when the cosmetic according to the present invention is used.

The alkylene oxide derivative to be used in the cosmetic according to the present invention is not particularly limited and is preferably a compound represented by the following Formula (1).

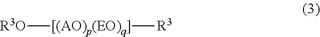

R³s each independently represent a hydrocarbon group having 1 to 4 carbon atoms or a hydrogen atom, and the ratio of the number of hydrogen atoms to the number of hydrocarbon groups of R³ is 0.15 or less and preferably 0.06 or less,
AO is an alkyleneoxy group having 3 or 4 carbon atoms,
EO is an ethyleneoxy group,
AO and EO each may be added in block form or at random,
p and q represent the average number of moles of AO and EO added, respectively,

$$\begin{matrix}1 \leq p \leq 70 , preferably 2 \leq p \leq 20 , \\ 1 \leq q \leq 70 , preferably 2 \leq q \leq 20 ,\end{matrix}$$

and
the ratio of EO to the total of AO and EO is 20 to 80 mass %).

Examples of the alkylene oxide derivative include
polyoxyethylene (10 mol) polyoxypropylene (10 mol) dimethyl ether,
polyoxyethylene (9 mol) polyoxypropylene (2 mol) dimethyl ether,
polyoxyethylene (14 mol) polyoxypropylene (7 mol) dimethyl ether,
polyoxyethylene (6 mol) polyoxypropylene (14 mol) dimethyl ether,
polyoxyethylene (15 mol) polyoxypropylene (5 mol) dimethyl ether,
polyoxyethylene (25 mol) polyoxypropylene (25 mol) dimethyl ether,
polyoxyethylene (9 mol) polyoxybutylene (2 mol) dimethyl ether,
polyoxyethylene (14 mol) polyoxybutylene (7 mol) dimethyl ether,
polyoxyethylene (10 mol) polyoxypropylene (10 mol) diethyl ether,
polyoxyethylene (10 mol) polyoxypropylene (10 mol) dipropyl ether,
polyoxyethylene (10 mol) polyoxypropylene (10 mol) dibutyl ether,
and
polyoxyethylene (36 mol) polyoxypropylene (41 mol) dimethyl ether.
Of these
polyoxyethylene (9 mol) polyoxypropylene (2 mol) dimethyl ether,
polyoxyethylene (14 mol) polyoxypropylene (7 mol) dimethyl ether, or
polyoxyethylene (36 mol) polyoxypropylene (41 mol) dimethyl ether
is preferable, and polyoxyethylene (14 mol) polyoxypropylene (7 mol) dimethyl ether is particularly preferable.

The amount of the alkylene oxide derivative which may be contained in the cosmetic according to the present invention is not particularly limited, but is preferably 0.01 to 10 mass % and more preferably 0.1 to 5 mass % based on the total mass of the cosmetic.

The cosmetic according to the present invention may contain components commonly used in the cosmetics other than the components described above. Examples of the other components include a powder component, a higher alcohol, a higher fatty acid, a surfactant except component (A1) or (A2), a moisturizing agent except component (D), a water-soluble polymer, a coating agent, a sequestering agent, a lower alcohol, a sugar, an amino acid, an organic amine, a polymer emulsion, a pH adjuster, a skin nutrient, a vitamin, an antioxidant, an antioxidant auxiliary, a flavor, and a colorant.

The formulation of the cosmetic according to the present invention is not particularly limited as long as it is an oil-in-water cosmetic. Note that, the oil-in-water cosmetic has a form prepared by dispersing oil droplet particles in water, but in order to maintain high transparency, the average particle diameter of the oil droplet particles contained in the cosmetic is preferably 70 nm or less.

The cosmetic according to the present invention is an oil-in-water cosmetic, and can be produced by any method selected from commonly known imagination methods for producing an oil-in-water cosmetic.

For example, a liquid prepared by mixing an oily ingredient therein is added to a solution prepared by mixing a surfactant and water, and the mixture is emulsified under high pressure by a high-pressure homogenizer to form an oil-in-water emulsified composition. The emulsification under high pressure is performed, for example, at a pressure of 50 MPa or more and preferably 100 MPa or more.

Herein, the emulsification method under high pressure is advantageous for producing an oil-in-water cosmetic having high transparency, but it is not always necessary to use the emulsification method under high pressure and the oil-in-water cosmetic can be produced by a commonly known production method such as a mixed nuclear reaction by adjusting a surfactant or dispersion conditions.

### Examples

Hereinafter, the present invention will be described more specifically with reference to Examples, but the present invention is not limited by these. Note that, unless otherwise specified, the content rate is expressed as mass % to the total mass of the cosmetic.

Oil-in-water emulsion cosmetics having compositions shown in the following Table 1 were produced and evaluated for the following items. The results of them are also shown in Table 1.

### [Transparency]

An L value was measured using a color difference meter (Spectrocolorimeter SE7700 (product name), manufactured by Nippon Denshoku Industries Co., Ltd.) at 30° C. based on purified water. From the measured L value, evaluation was performed in accordance with the following criteria.
A: L value is 35 or more
B: L value is less than 35

### [Stability]

Samples were stored at room temperature for 4 weeks, and changes were observed and evaluated in accordance with the following criteria.
A: No oil floating was observed on the surface
B: Extremely small amount of oil seemed to be floating on the surface
C: Oil floating spreads over the surface

### [Average Emulsified Particle Size]

The average size of emulsified particles was determined using a particle size measuring device (Zetasizer (product name), manufactured by Malvern Panalytical Ltd.).

### [Usability]

Each of the samples was applied to an arm or a face, and the degree of stickiness was evaluated in accordance with the following criteria.
A: Non-sticky
B: Sticky but acceptable
C: Very sticky

TABLE 1

| | | Example | | | | | | | | |
|---|---|---|---|---|---|---|---|---|---|---|
| | Component | 1 | 2 | 3 | 4 | 5 | 6 | 7 | 8 | 9 |
| A1 | Polyoxyethylene (20) | 3.5 | 3.5 | 3 | 3.5 | 3.5 | 3.5 | 3.5 | 0.35 | 5 |
| | polyoxypropylene (8) cetyl ether | | | | | | | | | |
| A1 | Beheneth-30 | | | | | | | | | |
| comparison | PEG-100 Hydrogenated castor oil | | | | | | | | | |
| | PPG-30 Buteth-30 | | | | | | | | | |
| | PEG/PPG-50/40 dimethyl ether | | | | | | | | | |
| A2 | Sodium stearoyl methyl taurate | 0.1 | 0.1 | 0.1 | | | | 0.1 | 0.01 | 0.1429 |
| | Na methyl cocoyl taurate | | | | 0.09 | | | | | |
| | Na taurine cocoyl methyltaurate | | | | | 0.08 | | | | |
| | Na stearoyl glutamate/ | | | | | | 0.1 | | | |
| B1 | Ethylhexyl methoxycinnamate | 7 | 7.5 | 7 | 7 | 7 | 7 | 7 | 0.7 | 10 |
| | Bis-ethylhexyloxyphenol | | 1.5 | | | | | | | |
| | methoxyphenyltriazine | | | | | | | | | |
| | Diisopropyl sebacate | | | | | | | | | |
| | Glyceryl (behenate/eicosanedioate) | | 0.2 | | | | | | | |
| B2 | Squalane | 3 | 2.8 | 3 | 3 | 3 | 3 | 3 | 0.3 | 4.2857 |
| | Dimethicone | | | | | | | | | |
| D | Dipropylene glycol | 8 | 8 | 8 | 8 | 8 | 8 | 8 | 0.8 | 11.429 |
| | Dynamite glycerin | 10 | 10 | 10 | 10 | 10 | 10 | 7 | 1 | 14.286 |
| | Phenoxyethanol | 0.5 | 0.5 | 0.5 | 0.5 | 0.5 | 0.5 | 0.5 | 0.05 | 0.7143 |
| | PEG/PPG-14/7 dimethyl ether | | | | | | | 3 | | |
| C | Water | Balance | Balance | Balance | Balance | Balance | Balance | Balance | Balance | Balance |
| | Total | 100 | 100 | 100 | 100 | 100 | 100 | 100 | 100 | 100 |
| | B/A1 | 2.86 | 3.43 | 3.33 | 2.86 | 2.86 | 2.86 | 2.86 | 2.86 | 2.86 |
| | A2/A1 | 0.029 | 0.029 | 0.033 | 0.026 | 0.023 | 0.029 | 0.029 | 0.029 | 0.029 |
| Evaluation | Transparency (L value) | 56.78 | 40.61 | 42.03 | 48.33 | 42.2 | 49.01 | 55.87 | 77.46 | 58.7 |
| | Transparency (qualitative) | A | A | A | A | A | A | A | A | A |
| | Stability | A | A | A | A | A | A | A | A | A |
| | Average emulsified particle size (nm) | 61.25 | 66.42 | 69.43 | 60.49 | 60.28 | 62.96 | 60.23 | 61.25 | 61.25 |
| | Usability | B | B | B | B | B | B | B | A | B |

| | | Example | Comparative Example | | | | | | |
|---|---|---|---|---|---|---|---|---|---|
| | Component | 10 | 1 | 2 | 3 | 4 | 5 | 6 | 7 |
| A1 | Polyoxyethylene (20) | 3.5 | 3.5 | | | | | 1.8 | 11 |
| | polyoxypropylene (8) cetyl ether | | | | | | | | |
| A1 | Beheneth-30 | | | 3.5 | | | | | |
| comparison | PEG-100 Hydrogenated castor oil | | | | 3.5 | | | | |
| | PPG-30 Buteth-30 | | | | | 3.5 | | | |
| | PEG/PPG-50/40 dimethyl ether | | | | | | 3.5 | | |
| A2 | Sodium stearoyl methyl taurate | 0.1 | | 0.1 | 0.1 | 0.1 | 0.1 | 0.1 | 0.1 |
| | Na methyl cocoyl taurate | | | | | | | | |
| | Na taurine cocoyl methyltaurate | | | | | | | | |
| | Na stearoyl glutamate/ | | | | | | | | |
| B1 | Ethylhexyl methoxycinnamate | 7 | 7 | 7 | 7 | 7 | 7 | 7 | 7 |
| | Bis-ethylhexyloxyphenol | | | | | | | | |
| | methoxyphenyltriazine | | | | | | | | |
| | Diisopropyl sebacate | 2 | | | | | | | |
| | Glyceryl (behenate/eicosanedioate) | | | | | | | | |
| B2 | Squalane | 1 | 3 | 3 | 3 | 3 | 3 | 3 | 3 |
| | Dimethicone | | | | | | | | |
| D | Dipropylene glycol | 8 | 8 | 8 | 8 | 8 | 8 | 8 | 8 |
| | Dynamite glycerin | 10 | 10 | 10 | 10 | 10 | 10 | 10 | 10 |
| | Phenoxyethanol | 0.5 | 0.5 | 0.5 | 0.5 | 0.5 | 0.5 | 0.5 | 0.5 |
| | PEG/PPG-14/7 dimethyl ether | | | | | | | | |
| C | Water | Balance | Balance | Balance | Balance | Balance | Balance | Balance | Balance |
| | Total | 100 | 100 | 100 | 100 | 100 | 100 | 100 | 100 |
| | B/A1 | 2.86 | 2.86 | 2.86 | 2.86 | 2.86 | 2.86 | 5.56 | 0.91 |
| | A2/A1 | 0.029 | 0.000 | 0.029 | 0.029 | 0.029 | 0.029 | 0.056 | 0.009 |
| Evaluation | Transparency (L value) | 38.01 | 19.34 | 26.64 | 7.51 | 6.22 | 33.36 | 7.54 | 86.34 |
| | Transparency (qualitative) | A | B | B | B | B | B | B | A |
| | Stability | B | A | C | C | — | A | — | A |
| | Average emulsified particle size (nm) | 65.67 | 62.2 | 71.51 | 90.28 | 102.1 | 80.99 | 93.42 | 34.94 |
| | Usability | B | B | B | B | B | B | A | C |

From the obtained results, it is found that a cosmetic containing a combination of the surfactants specified and a polar oil has high transparency and excellent stability. In contrast, it is found that a cosmetic not containing either one of the two surfactants specified in the present invention is inferior in either transparency or stability.

1. An oil-in-water cosmetic comprising:
(A1) a first surfactant represented by the following formula (1): 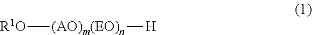
wherein
R¹ is a hydrocarbon group having 16 to 18 carbon atoms,
AO is an alkyleneoxy group having 3 or 4 carbon atoms,
EO is an ethyleneoxy group,
AO and EO are each added in block form,
m and n represent average numbers of moles of AO and EO added, respectively,
$$\begin{matrix}m > 4 , \\ n > 10 , and \\ n > m ,\end{matrix}$$
(A2) a second surfactant selected from ionic surfactants,
(B1) a polar oil, and
(C) water,
wherein
a content rate of component (B1) to a total mass of oil component (B) contained in said oil-in-water cosmetic is 50 mass % or more, and
a ratio B/A1 of the total mass of oil component (B) to a total mass of component (A1) contained in said oil-in-water cosmetic is 1 to 5.

2. The oil-in-water cosmetic according to claim 1, wherein component (A2) is a compound containing two carboxyl groups.

3. The oil-in-water cosmetic according to claim 1, wherein component (A2) comprises a structure represented by the following Formula (2): 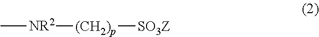
wherein
R² is H or a hydrocarbon group having 1 or 2 carbon atoms,
Z is H, Na, K, or NR^{a}₄ (wherein R^{a}s are each independently H, CH₃, C₂H₅, CH₂OH or CH₂CH₂OH), and
p is 1 to 3.

4. The oil-in-water cosmetic according to claim 1, wherein at least a part of component (B1) is a UV absorber.

5. The oil-in-water cosmetic according to claim 1, wherein a content rate of component (A1) to a total mass of said oil-in-water cosmetic is 0.1 to 15 mass %.

6. The oil-in-water cosmetic according to claim 1, wherein a content rate of component (A2) to the total mass of said oil-in-water cosmetic is 0.01 to 1 mass %.

7. The oil-in-water cosmetic according to claim 1, wherein a ratio A2/A1 of a total mass of component (A2) to the total mass of component (A1) contained in said oil-in-water cosmetic is 0.01 to 0.05.

8. The oil-in-water cosmetic according to claim 1, further comprising a polyhydric alcohol (D).

9. The oil-in-water cosmetic according to claim 8, wherein a content rate of component (D) to the total mass of said cosmetic is 3 to 30 mass %.

10. The oil-in-water cosmetic according to claim 1, wherein oil droplet particles contained in said oil-in-water cosmetic have an average particle diameter of 70 nm or less.
